(19) United States
(12) Patent Application Publication
(10) Pub. No.: US 2008/0125689 A1
(43) Pub. Date: May 29, 2008

(54) OXIMETRY AND CAPNOGRAPHY SYSTEM APPLIED TO EXTRACORPOREAL CIRCULATION - ECC - PROCEDURE

(76) Inventors: Osvaldo NOGUEIRA SANCHES, San Paulo (BR); Nadia Maria ROCHA MENDES, San Paulo (BR); Gilberto DE OLIVEIRA SCUCIATO, San Paulo (BR)

Correspondence Address: SUGHRUE MION, PLLC, 2100 PENNSYLVANIA AVENUE, N.W., SUITE 800, WASHINGTON, DC, 20037, US

(21) Appl. No.: 11/923,101

(22) Filed: Oct. 24, 2007

(30) Foreign Application Priority Data
Nov. 27, 2006 (BR) 0605449-8

(51) Int. Cl. A61M 1/36

(52) U.S. Cl. 604/6.14; 422/45

(57) ABSTRACT

The present invention refers to an oximetry and capnography system applied to extracorporeal circulation procedure, more specifically refers to an oximetry and capnography monitoring system associated to gas analyzer applied to ECC, in which a mechanical device (1) based on a tubular circuit (2) is foreseen, and connectors (3) that when duly installed in the equipment operating in ECC have as main purpose to monitor the operation of the gas blender, analyze the oxygenation chamber performance, and check CO₂ outflow from the oxygenation chamber in such a way as to prevent collapse within the said oxygenation chamber and therefore, being efficient throughout the ECC procedure.

### TECHNICAL FIELD

The present invention refers to an oximetry and capnography system applied to extracorporeal circulation procedure, more specifically refers to an oximetry and capnography monitoring system associated to gas analyzer applied to ECC, in which a mechanical device is foreseen based on a tubular circuit and connectors that when duly installed in the equipment operating in ECC have as main purpose to monitor the operation of the gas blender, analyze the oxygenation chamber performance, and check CO₂ outflow from the oxygenation chamber in such a way as to prevent collapse within the said oxygenation chamber and therefore, being efficient throughout the ECC procedure.

### TECHNIQUE BASIS

The extracorporeal circulation—ECC—is a procedure carried out in most of the cardiac surgeries by which the machine performs a full cardiopulmonary bypass, i.e., diverts blood from the vena cava to a reservoir and reinfuses it back to aorta after artificial oxygenation, temporarily replacing heart pumping and lung ventilation functions.

Technical difficulties to maintain this kind of mechanical support are largely known, which range from lack of trained personnel, known as perfusionists, who basically properly monitor this therapeutic feature.

In general, the extracorporeal procedure uses cannulae to maintain circulation, which are inserted by means of sternal thoracotomy by which venous blood is diverted from heart to the extracorporeal circulation circuit by introducing cannulae into the right atrium or venae cavae, with one cannula directed to the vena cava superior and the other directed to the vena cava inferior. Oxygenated blood, on its time, passes through an arterial pump and comes back to the patient by means of a cannula placed into the ascending aorta (venous-arterial short-circuit). The cannula gauge varies according to the patient's weight. The cannulae system is then kept within the thorax and this is closed, allowing only for the passage of the cannulae to the outside.

The arterial flow is kept between 40 to 60 ml/kg/min for the adult system, 80 to 110 ml/kg/min for the child system, and 100 to 150 ml/kg/min for the neonate system, at a temperature of 37° C. or 2.2 to 2.4 liters/kg/square meter body surface, with this flow being lowered or increased according to the mean blood pressure fluctuation and kept within the limits of the patient's weight.

As previously stated, the perfusionist participates in the surgery by monitoring data provided by the equipment during ECC, with the purpose of correcting oxygen and CO₂ dosage in view of the ongoing analysis of the data recorded by the gas analyzer. Most of the perfusionists work based on their own professional expertise by visually checking the blood type leaving the patient and the blood type returning to the patient, which is called by the professionals as “arterial-venous difference”.

One of the equipment responsible for ECC is the gas mixer, known as blender, which performs oxygen and compressed-air dosage in relation to the patient's gasometry; said blender handles the oxygen concentration levels and must be monitored by the perfusionist in order to keep patient's oxygenation throughout the whole surgery.

Any data reading or analysis error provided by the equipment analyzer may lead to patient's death and most of the times, among other responsibilities, the staff must have the equipment serviced in order to correct faults such as membrane deviation and gas unbalance, among others.

The gas blender may present two faults that may be detected only after gasometry result, as follows:
a) Pressure unbalance (compressed air and oxygen) within the gas blender:
   when compressed-air pressure is higher than oxygen pressure the obtained oxygen concentration will be lower than the one foreseen by the perfusionist, causing hypoxia to the patient;
   when oxygen pressure is higher than compressed-air pressure, oxygen concentration will be higher than the one foreseen by the perfusionist, causing blood hyperoxigenation and gas embolism;
b) Gas blender membrane deviation outside its normal position, giving an unadjusted concentration and causing the same troubles of the above mentioned faults.

In any circumstance, however, whether apparatus fault or reading error by the perfusionist, the consequences fall upon the patient.

Some clinical sites use well-defined assistance protocols for ECC maintenance for children or adults until heart function is recovered. When followed by the perfusionists, these protocols enable addressing patients, once changes arising out of anticoagulation, heart and lung arrest during ECC, and blood gasometry must be early identified through sophisticated and fast laboratory techniques. However, in such cases, the correct reading of data provided by the equipment is also full perfusionist's responsibility.

A few surgical rooms use imported equipment with sensors installed in the extracorporeal circuit, which send steady signals to the gas analyzer equipment, which is also imported, allowing for the correct data reading by the perfusionist. However, such equipment and sensors are highly expensive and almost unaffordable for 90% of the Brazilian hospitals.

### BRIEF DESCRIPTION OF THE INVENTION

With the purpose of eliminating the issues presented by the state of the technique, the applicants developed a system that by means of oximetry and capnography associated to the gas analyzed, monitors the operation of such equipment during ECC, enabling the identification of any disturbance during the procedure, correctly solving the problem, and preventing great disorders during ECC.

The said system comprises a derived circuit made up by flexible tubes interconnected by a connection having a regulating device, and one of the tubes takes a sample of the gas entering the oxygenator to the gas analyzer and consistently checks its actual concentration, which is simultaneously checked against the existing concentration within the gas mixer (blender).

The derived circuit enables the analysis both of the gas outflow and the gas inflow from and to the oxygenator, and makes an analysis of the oxygenation performance. For that, in view of the several existing models, the oxygenator must have a single gas exit, i.e., the remaining ones must be closed for the result to be achieved.

In addition to analyzing oxygenator oximetry, the system enables the control of CO₂ outflow from the oxygenator by means of capnography, this CO₂ removal being related to the gas flow relative to the arterial flow.

Blood gasometry results obtained upon collection will remain unchanged, provided that the difference of inflow and outflow gases to and from the oxygenator and CO₂ removal are kept stable during ECC.

In summary, the oximetry and capnography system analyzes the gas blender and the gas exchange system efficiency within the oxygenation chamber of the single-exit oxygenator through the comparison of the gas entering the oxygenator and the gas exiting the oxygenator.

### DESCRIPTION OF THE DRAWINGS

As a complement to the present description in order to achieve a better understanding of the present invention features, and according to its practical intended performance, a set of drawings are attached to this description, where the following is represented as an example, being not limited.

FIG. 1 shows the schematic drawing of the oximetry and capnography system applied to ECC, which is made up by the tubing and connector circuit installed in the conventional oximetry and gas analyzer equipment.

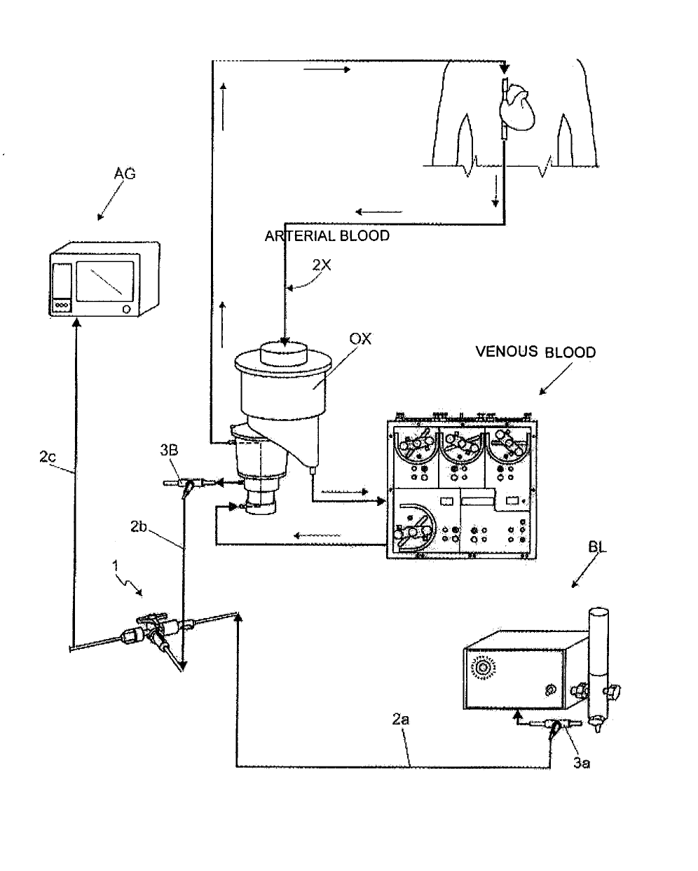

### DETAILED DESCRIPTION OF THE INVENTION

With reference to the drawings, the present invention refers to the “OXIMETRY AND CAPNOGRAPHY SYSTEM APPLIED TO EXTRACORPOREAL CIRCULATION—ECC—PROCEDURE, the present system more precisely comprises a mechanical device (1) made up by a flexible tube (2) and connector (3) circuit that perform consistent monitoring of ECC, with the connector (3A) that connects to the tube (2A) taking a sample of the gas entering the oxygenator (OX) to the mechanical device (1) which, connected to the tube (2C), takes the said sample to the gas analyzer (AG) consistently checking its actual concentration, which is simultaneously checked with the existing concentration within the gas blender (BL).

And through the connector (3B) located at the oxygenator (OX) exit, and through the tube (2B), it takes a sample of the gas exiting the oxygenator (OX) to the mechanical device (1) that takes it through the tube to the gas analyzer (AG) checking CO₂ outflow from the oxygenator (OX) due to venous blood change to arterial blood.

Venous blood leaves the heart through the tubes (2X), passes through the oxygenator (OX) from which it is sent to the ECC machine (MCEC) from which it returns to the oxygenator (OX) through a propelling pump to be oxygenated and changed to arterial blood and returned to the patient through the tube (2Y), making an extracorporeal circulation—ECC—, that is, a circulation outside the body.

The derived circuit (1) enables control of the CO₂ outflow from the oxygenator (OX) by means of capnography and this CO₂ removal is related to the gas flow relative to the arterial flow.

By means of oximetry and capnography, the said system analyzes the gas blender (BL) and gas exchange system efficiency within the oxygenation chamber of the single-exit oxygenator through the comparison of the gas entering (E) the oxygenator (OX) with the gas exiting (S) the oxygenator (OX).

Despite the detailed invention, it is important to understand that its application is not limited to the details and steps described here. The invention is capable of other modalities and of being used or performed in a variety of modes. It must be understood that the terminology employed here is for description and not limitation purposes.

1.) “OXIMETRY AND CAPNOGRAPHY SYSTEM APPLIED TO EXTRACORPOREAL CIRCULATION—ECC—PROCEDURE”, the present system is more precisely featured to comprise a mechanical device (1) made up by flexible tube (2) and connector (3) circuit which by means of oximetry and capnography analyzes the gas blender (BL) and the gas exchange system efficiency within the oxygenation chamber of the single-exit oxygenator (OX) through the comparison of the gas entering (E) the oxygenator (OX) relative to the gas exiting (S) the oxygenator (OX).

2.) “OXIMETRY AND CAPNOGRAPHY SYSTEM APPLIED TO EXTRACORPOREAL CIRCULATION—ECC—PROCEDURE”, according to the 1^{st} claim, featured by the derived circuit 1) to monitor, by means of oximetry, ECC through a tube (2A) that takes a sample of the gas entering the oxygenator (OX) to a gas analyzer (AG) and consistently checks its actual concentration, which is simultaneously checked with the existing concentration of the gas blender (BL) and through the tube (2B) that takes a sample of the gas exiting the oxygenator (OX) to the gas analyzer (AG) and checks CO₂ outflow from the oxygenator (OX).

3.) “OXIMETRY AND CAPNOGRAPHY SYSTEM APPLIED TO EXTRACORPOREAL CIRCULATION—ECC—PROCEDURE”, according to the 1^{st} claim, featured by the derived circuit (1) to monitor CO₂ outflow from the oxygenator (OX) by means of oximetry, with this CO₂ removal being related to the gas flow in relation to the arterial flow.
